(19) United States
(12) Patent Application Publication
(10) Pub. No.: US 2007/0293949 A1
(43) Pub. Date: Dec. 20, 2007

(54) Interior connecting interbody cage insertional tools, methods and devices

(76) Inventors: Anthony A. Salerni, Bedford, NH (US); Marc M. Peterman, Austin, TX (US); Jeffrey D. Moore, Olive Branch, MS (US)

Correspondence Address: KRIEG DEVAULT LLP, ONE INDIANA SQUARE, SUITE 2800, INDIANAPOLIS, IN, 46204-2709, US

(21) Appl. No.: 11/731,333

(22) Filed: Mar. 30, 2007

Related U.S. Application Data
(63) Continuation of application No. PCT/US05/37649, filed on Oct. 10, 2005.
(60) Provisional application No. 60/617,280, filed on Oct. 8, 2004.

(51) Int. Cl. A61F 2/44

(52) U.S. Cl. 623/17.16

(57) ABSTRACT

Interior connecting interbody cage insertional tools, methods and devices are provided wherein the same can be utilized for making placement of interbody cages more accurate, safer and less likely to violate the structural integrity of the interbody cage while providing support to a leading end of the cage as the leading end re-capitulates or distracts the disc space as the cage is inserted.

### CROSS-REFERENCE TO RELATED APPLICATIONS

This application is a continuation of PCT Application No. PCT/US2005/037649 filed on Oct. 10, 2005, which claims priority to and the benefit of the filing date of Provisional Application Ser. No. 60/617,280 filed on Oct. 8, 2004, each of which is incorporated herein by reference in its entirety.

### BACKGROUND

1. Field of the Invention

The present invention relates generally to interbody spinal systems and more specifically it relates to interior connecting interbody cage insertional tools, methods and devices for making placement of interbody cages more accurate, safer and less likely to violate the structural integrity of the interbody cage.

2. Description of the Related Art

It can be appreciated that interbody insertional tools have been in use for years. Typically, interbody insertional tools are comprised of insertion tools used to introduce and properly position various types of devices into the interbody spaces in the spinal column. Most spinal interbody cages are inserted in a direction parallel to their long axis. Interbody insertional tools provide the forces necessary for the insertion and proper placement of interbody cages. These insertional tools connect to their respective interbody devices by the application of some type of force generated by the insertional tool to the outside surfaces of the interbody device or cage. Two basic methods of contact include a screw, cam or snap to lock the inserter to its device and inserters that have been fashioned to function like elongated vises or pliers that apply their gripping forces to the outside lateral surfaces of their respective interbody devices.

One potential problem with conventional interbody insertional tools is that the most commonly employed means to connect the inserter to its device is relatively weak and may break if substantial forces are applied during the insertion process. This weak connection can result in suboptimal placement in the interbody space or even a loss of control of the interbody device during placement, increasing the time and complication of the surgery. Another potential problem with conventional interbody insertional tools is that insertional forces are applied limited surfaces of the interbody device. This could lead to deformation, fatigue and other forces being exerted on the device. Another potential problem with conventional interbody insertional tools is that the vise-like inserters reduce visibility because of their bulk.

While these devices may be suitable for the particular purpose to which they address, they are not as suitable for making placement of interbody cages more accurate, safer and less likely to violate the structural integrity of the interbody cage. In these respects, the interior connecting interbody cage insertional tools, methods, and devices according to the present invention, substantially depart from the conventional concepts and designs of the prior art, and in so doing provides an apparatus primarily developed for the purpose of making placement of interbody cages more accurate, easier and less likely to violate the structural integrity of the interbody cage.

### SUMMARY

The present invention provides interior connecting interbody cage insertional tools, methods and devices wherein the same can be utilized for making placement of interbody cages more accurate, safer and less likely to violate the structural integrity of the interbody cage while providing support to a leading end of the cage as the leading re-capitulates or distracts the disc space as the cage is inserted.

In one aspect, the insertional tool includes a coupling member to engage a trailing end of the cage and an inner shaft extending from the coupling member from the trailing end of the cage and through the interior of the cage to engage the leading end of the cage.

In a further aspect, the trailing end of the cage includes a through-opening sized to accept a portion of an insertional tool and a leading end hole to engage an inner shaft of the tool extending distally from the coupling portion. The through-opening includes at least one of a height that is greater than one half the height of the cage at the trailing end and a width that is greater than one-half a width of the cage at the trailing end. The trailing end opening size allows maximum surface area contact between the coupling member of the insertion instrument and the trailing end of the cage within the opening, facilitating the application of insertion forces while reducing the torsional and bending stresses exerted on the cage by the insertional tool.

In another aspect, an interbody fusion device includes a cage body with a leading end having a rounded nose extending between convexly curved upper and lower surfaces. The body includes a hollow interior opening at the upper and lower surfaces of the cage body. Opposite sidewalls extend from the leading end nose to a trailing end. The trailing end includes a through-opening in communication with the hollow interior. The through-opening includes a generally rectangular shape on a longitudinal axis of the cage body, and the leading end nose includes a circular hole extending along the longitudinal axis and in communication with the hollow interior of the cage body.

These and other aspects will be apparent from the following description.

### BRIEF DESCRIPTION OF THE FIGURES

FIG. 1 is a perspective view of one embodiment insertional tool that is assembled.

FIG. 2 is an exploded perspective view of the insertional tool of FIG. 1.

FIG. 3 is an enlarged exploded view of the insertional tool of FIG. 1 that has been sectioned approximately across its midsection.

FIG. 4 is a perspective view of distal end of the insertional tool engaged to an interbody implant device.

FIG. 5 is a perspective view of an interbody implant device.

FIGS. 6-8 are various perspective views of another embodiment interbody implant device.

FIG. 9 is a perspective view of a distal end portion of another embodiment insertional tool.

FIG. 10 is a perspective view of another embodiment implant.

FIG. 11 is a diagram showing a surface configuration of the implant of FIG. 10.

FIG. 12 is a transverse sectional view of another embodiment of the implant of FIG. 10.

FIG. 13 is a perspective view of a distal portion of another embodiment insertional tool.

FIG. 14 is a partial sectional view of a portion of the insertional tool of FIG. 13.

FIG. 15 is an elevation view of a locking member of the insertional tool of FIG. 14.

FIG. 16 is a partial sectional view of a portion of the insertional tool of FIG. 13.

### DESCRIPTION OF THE ILLUSTRATED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Insertional tools, methods and devices for interbody stabilization are provided that facilitate control and positioning of the leading end of the cage to be inserted. Placement of interbody cages can be more accurate, efficient, and less likely to violate the structural integrity of the interbody cage. Insertional tools and cages are configured so that the inserter tips fit into and positively lock with the interior of the specific interbody cage that it might be adapted to and designed to drive. The insertional tools and cages allow absolute and positive directional control over the spinal interbody device during the insertion process for precise positioning thereof. The insertional tools and cages distribute the insertional forces more uniformly over the interbody device, including internal wall surfaces, to reduce the risk that the interbody device will suffer fracture and deformation. The insertional tools and cages minimize interference with visibility during the insertional process. The insertional tools and cages can further facilitate removal of the specific interbody device to which it is adapted.

The Figures illustrate various embodiments of insertional tools. The insertional tools can comprise an inserter tip, body and locking system. The inserter tip may be either an integral part of the body of this interbody insertional tool or it may be a component that is attached to the body. The general shape of the inserter tip can be proscribed by the dimensions of the interior space of the interbody cage to which it has been adapted. Functionality of the tool can be a consequence of the internal position of its tip in its specific interbody cage. The inserter tip can be hollow in some fashion so it might serve as a housing for an actuator for the locking mechanism. The end of the inserter tip may be fashioned to mate with the back side of the leading surface of the interbody cage to which it is attached so that a portion of the insertional force may be transmitted to this area. The inserter tip may also utilize a buttress that is fashioned to mate with the back of the trailing surface of the interbody cage to which it is attached. This buttress may be fashioned to precisely engage this surface so that insertional forces may be more uniformly distributed over the surface of the interbody device.

The body of the insertional tool can be comprised of a handle and a shaft, which can be hollow. This hollow space directly communicates with the hollow space in the inserter tip. Together, these spaces define a locking system access channel. The handle is roughly cylindrical and may have ridges for better control. The shaft is roughly cylindrical in shape and it is mated to the handle and the inserter tip so that the three components behave in unison. The bulk of the locking system is, for the most part, housed in the locking system access channel in the body and tip of the insertional tool. The system can be comprised of a lock knob that protrudes from the handle, a lock shaft that resides completely within the locking system access channel and a lock or lock actuator that either is contained within or protrudes from part of the inserter tip. The exact nature and shape of the lock or lock actuator can be determined by the specific type of interbody device that is being used.

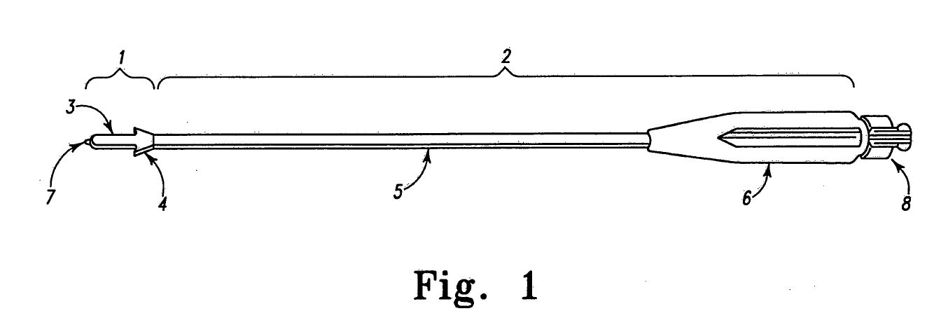

FIG. 1 is a view of one embodiment of the insertional tool. It has three major components: the inserter tip 1, which is comprised of the cage adapter 3 and the inserter tip buttress 4; the inserter body 2 which is comprised of the inserter shaft 5, and handle 6; and the locking system of which the lock actuator 7, and the lock knob 8 can be seen. The specific size and shape of the cage adapter will vary according to the specific requirements of the interbody cage that it is designed to fit.

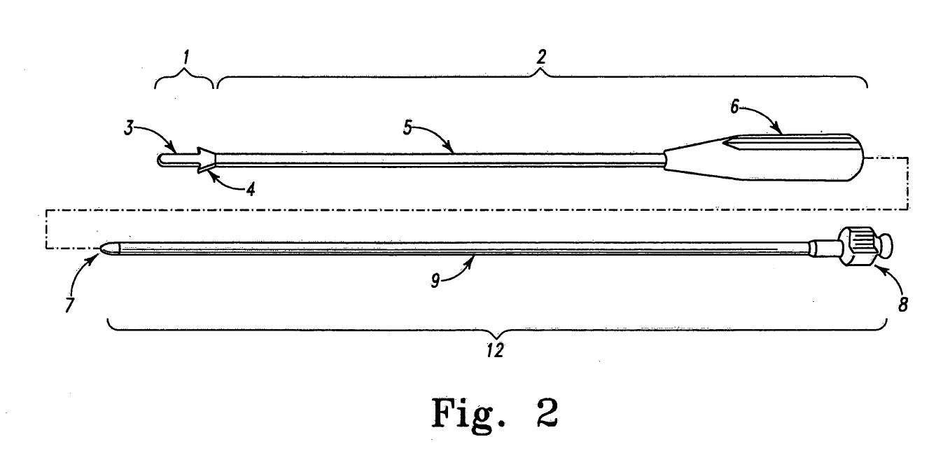

FIG. 2 is a disassembled view of the entire device of FIG. 1. The cage adapter 3 and the inserter tip buttress 4 of the inserter tip 1 and the inserter shaft 5 and handle 6 of the inserter body 2 are shown above. The lock actuator 7, lock shaft 9 and the lock knob 8 of the locking system 12 are shown below.

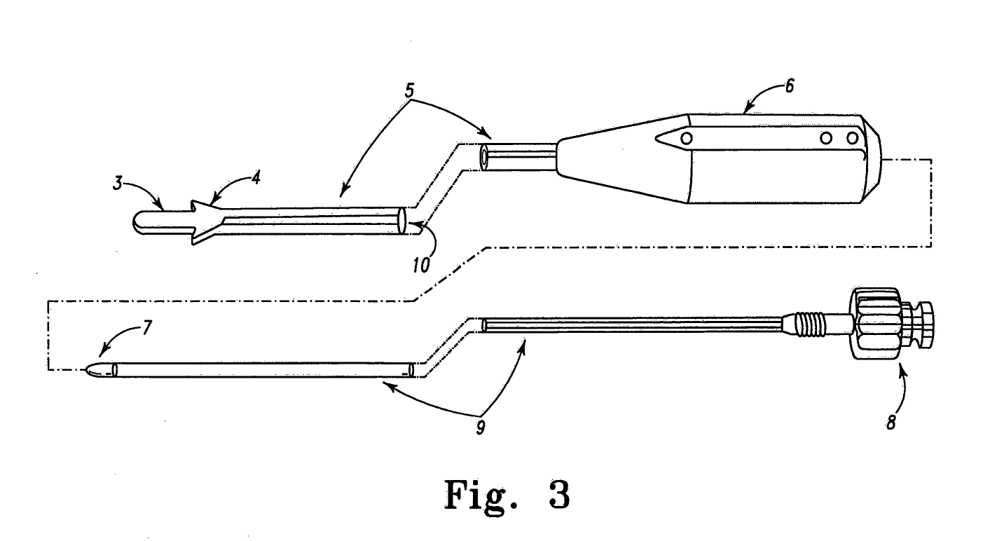

FIG. 3 is an enlarged view of the inserter tip, inserter body and locking system where a section of the shafts of each has been cut away at approximately mid-instrument. This cross section through the inserter shaft shows the lock access channel 10 that runs the length of the inserter tip and body and houses the lock shaft. Assembly of the insertional tool requires the lock actuator 7 to be inserted into the lock access channel 10 that opens out of the end of the handle. The locking system is slid down the length of the lock access channel 10 so that the lock actuator 7 protrudes from the inserter tip and the lock knob 8 abuts the end of the inserter handle 6.

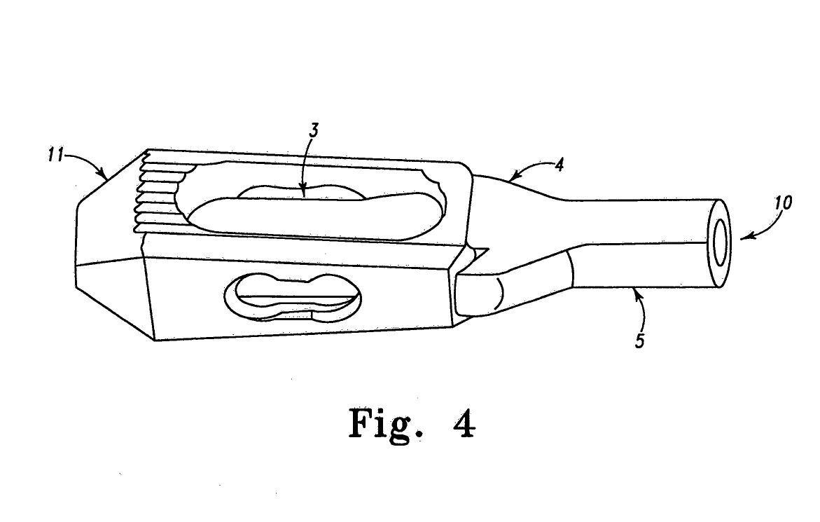

FIG. 4 is an operational view of the inserter tip end of the insertional tool. The cage adapter 3 is shown inside one embodiment interbody cage 11. Cage 11 precisely mates to the inserter tip buttress 4, which is uniformly tapered to join the inserter shaft 5. The lock access channel 10 is also depicted.

The cage adapter 3 of the inserter tip 1 is the portion of the interior connecting interbody cage insertional tool that fits securely into the interior of the interbody cage that it is designed to implant. The inserter tip buttress 4 is the portion of the tip that remains outside the interbody device and is connected to the inserter shaft 5. The end dimensions and shape of the buttress will generally, though not necessarily, be a mirror image of the trailing surface of the specific interbody device against which it abuts. Because many interbody devices have interior cross sections that are roughly rectangular, the cage adapter 3 of the inserter tip 1, is shown as box-like in FIGS. 1-4. It is understood however, that the interior shapes of interbody cages can exhibit substantial variability. The range of shapes that the inserter tip will be manufactured in will therefore reflect this variability. It is expected that in some situations it would be advantageous for the inserter tip to be frame-like or have openings as opposed to being solid. Movable components might also be added to the inserter tip to aid in securing it to its interbody device or help in the insertion process. The inserter tip could be modified so that it serves to lock and unlock it self to the interior of its interbody cage when the appropriate forces are transmitted to it through the shaft and handle of the body of the inserter.

The body 2 is comprised of the handle 6 and the inserter shaft 5. The handle is roughly cylindrical and may be grooved to improve the grip of the user. The shaft is cylindrical, or roughly so. The diameter of the inserter shaft is considerably smaller than its specific interbody device to aid visualization around it during the insertional process. Although it remains uniformly narrow along its midsection, the inserter shaft is tapered outward at its end so that the transition to the inserter tip buttress 4 is smooth. The smooth transition eliminates edges that could potentially damage anatomic structures. The handle of the device could be set off the line described by the long axis of the shaft to aid in visualization of interbody cage placement.

The locking system 12, as shown in FIGS. 2-3, is comprised of the lock knob 8, the lock shaft 9 and the lock actuator 7. The lock actuator depicted in FIGS. 1-3 is a simple screw thread mechanism where the lock actuator is screwed into the end of the interbody cage to which it has been adapted. The lock knob 8 projects from the handle 6 and is directly connected to the lock shaft 9 which is cylindrical or roughly so. As the lock knob 8 is rotated, the lock shaft 9 turns, engaging or disengaging the lock actuator 7 depending on the direction of the rotational force applied. The lock knob functions as a switching mechanism which when force is applied by the user the lock shaft moves in a way that activates the lock actuator.

The lock knob does not necessarily need to provide a rotational force to the shaft. Alternate switching mechanisms that cause the lock shaft to slide in and out of the lock access channel may also be used. The lock shaft could be comprised of a number of smaller subcomponents that are mechanically or hydraulically connected to the lock knob or alternate switching mechanism. The lock actuator may remain completely housed within the lock access channel inside the inserter tip. With this type of configuration the lock actuator might activate alternate locking mechanisms for the purpose of securing the inserter tool to its interbody cage. In other cases the inserter tip may also serve as the lock actuator. In this situation, the force used to activate the lock would be transmitted to the modified inserter tip by way of the body shaft and handle.

The lock shaft 9 resides within the lock access channel 10. The lock knob 8 protrudes from the handle 6 while the lock actuator 7 protrudes from the end of the inserter tip 1. The design of certain interbody cages could eliminate the need for a locking mechanism to be housed within the body and inserter tip. Therefore, the inserter tip itself could be fashioned into a locking mechanism or alternatively the lock may be placed external to the body of the inserter.

FIG. 4 demonstrates how this embodiment of the insertional tool will interact with a typical interbody cage. The cage adapter 3 of the inserter tip 1 is inserted into the trailing end opening of the interbody cage for which it has been modified to fit. The locking knob 8 is rotated in a clockwise direction so that the threaded screw-like lock actuator 7 engages the threaded hole in the tip of the interbody cage. As the locking mechanism is tightened the trailing surface of the leading edge and the trailing surface of the trailing edge of the interbody cage is drawn securely against the front edge of the cage adapter 3 and the buttress 4 of the inserter tip 1, respectively. Once secured, the interbody cage and the insertional tool function as a single entity. The handle 6 is used to position the leading edge of the interbody cage in proximity to its target. The insertional force is applied to the end of the handle 6 and transmitted in the direction along the length of the inserter shaft 7 to drive the interbody device to its proper target area. The orientation of the handle 6 may be varied to keep the path of the interbody device along its proper trajectory. Once the cage is in position, the locking mechanism is deactivated and the inserter tool is removed leaving the cage behind.

FIG. 5 is directed to an interbody fusion device 100 with a body 102 having a tapered leading insertion end 104. Body 102 includes parallel sidewalls 105, and a trailing end 106. An interior cavity defines a hollow interior 108 extending between and opening at upper and lower surfaces 110, 112. Sidewalls 105, 107 each include a hole 114 (only shown in sidewall 105), and a number of grooves 116 in the upper and lower surfaces 110, 112 form projections or ridges 118 that facilitate engagement of the device 100 with the adjacent vertebral endplates. The aggressively tapered insertion end 104 can be convexly curved to provide a rounded nose between upper and lower surfaces 110, 112 to facilitate insertion of device 100 into a collapsed spinal disc space. Device 100 can recapitulate the spinal disc space when inserted therein by contacting and separating the bony material of the adjacent vertebral bodies as it is inserted. Accordingly, it is not necessary to maintain distraction of the disc space to insert device 100, although providing distraction is not precluded. Further details regarding device 100 and instrumentation associated therewith are provided in U.S. patent application Ser. No. 10/766,167 filed on Jan. 28, 2004, (published Oct. 19, 2004 under U.S. Patent Application Publication No. US 2004-0162616) which is incorporated herein by reference in its entirety.

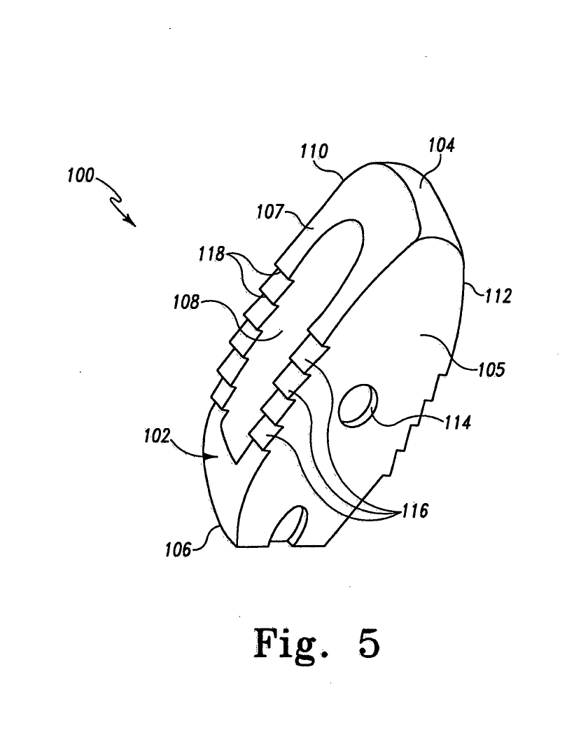

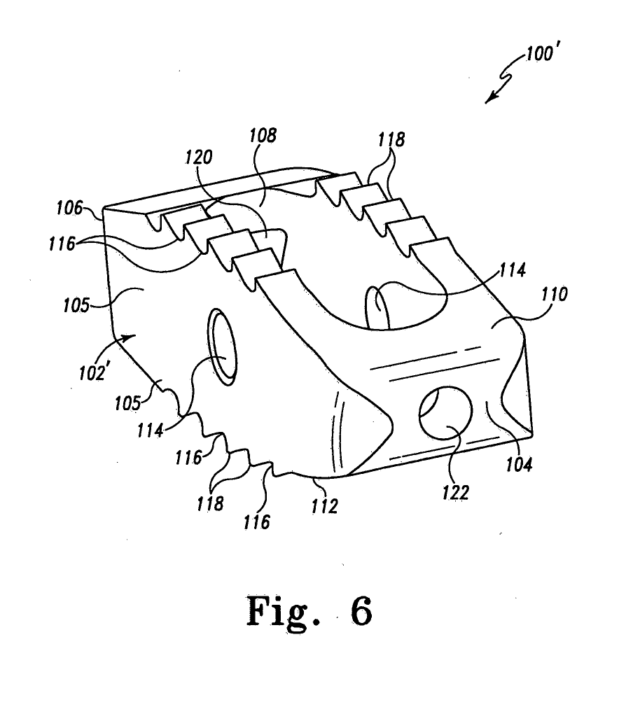

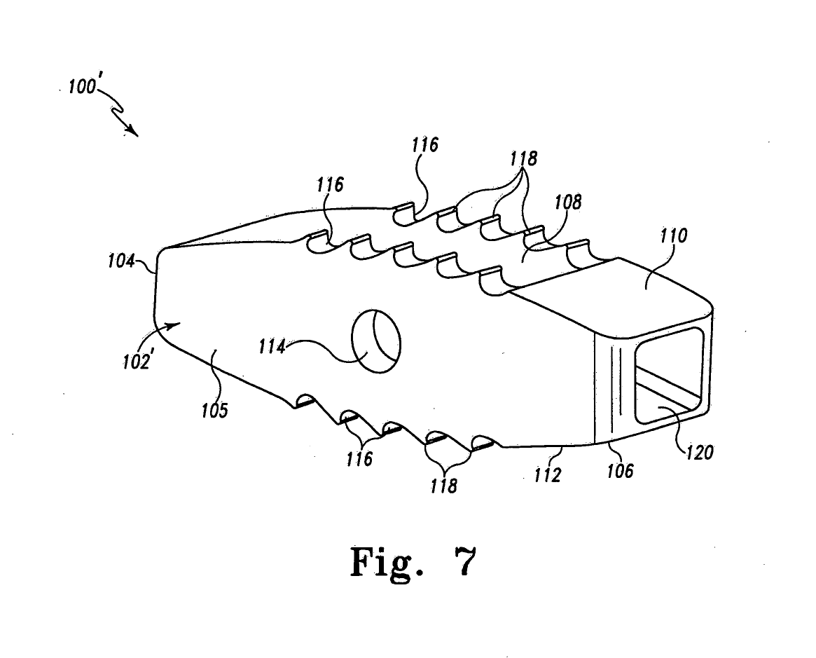

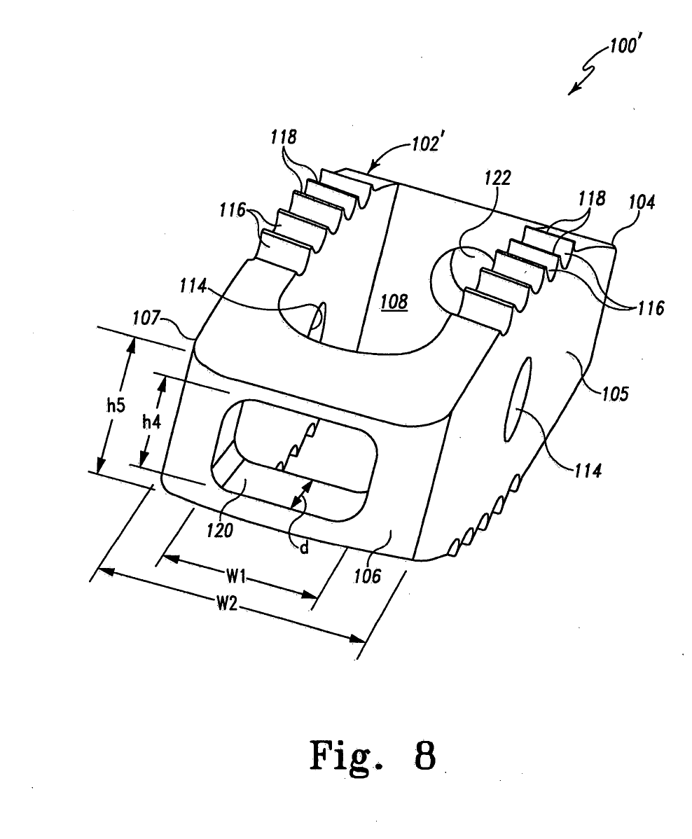

FIGS. 6-8 illustrate a variant of the device of FIG. 5 in which interbody device 100′ is provided with a cage body 102′ having an enlarged through-opening 120 at trailing end 106 and a hole 122 at lead end 104 to engage a coupling assembly of an insertional tool. Through-opening 120 and hole 122 communicate with hollow interior 108, and are aligned along and extend along the longitudinal axis of body 102. Hole 122 can include a circular cross-sectional shape and is elongated to extend completely through leading end 104. Hole 122 intersects the convexly curved nose of leading end 104, providing insertional tool control of the cage body 102′ along its entire length. This facilitates repositioning of cage body 102′ in the disc space in order to find optimal placement of the convexly curved upper and lower surfaces 110, 112 relative to the concavely curved vertebral endplates while minimizing stresses exerted on trailing end 106.

Through-opening 120 includes a generally rectangular shape that occupies at least one half of the area of trailing end 106. In one embodiment, through-hole 120 includes a width w1 and trailing end 106 includes a width w2 between sidewalls 105, 107. Width w1 can be at least one half of the width w2. Through-opening 120 can also include a height h4 in the direction between upper and lower surfaces 110, 112. Trailing end 106 includes a height h5 between upper and lower surfaces 110, 112. Height h4 can be at least one half of the height h5. The larger through-opening dimensions provide increased surface area about through-opening 120 for contact by a coupling assembly of an insertion tool, distributing the insertional forces about the surface area and positioning the insertional forces adjacent to the outer portions of the cage body.

Through-opening 120 further includes a depth d at trailing end 106 that corresponds to the wall thickness of cage body 102′ at trailing end 106. Through-opening 120 and hole 122 can provide additional bone growth openings when the coupling assembly of the insertional tool is disengaged therefrom.

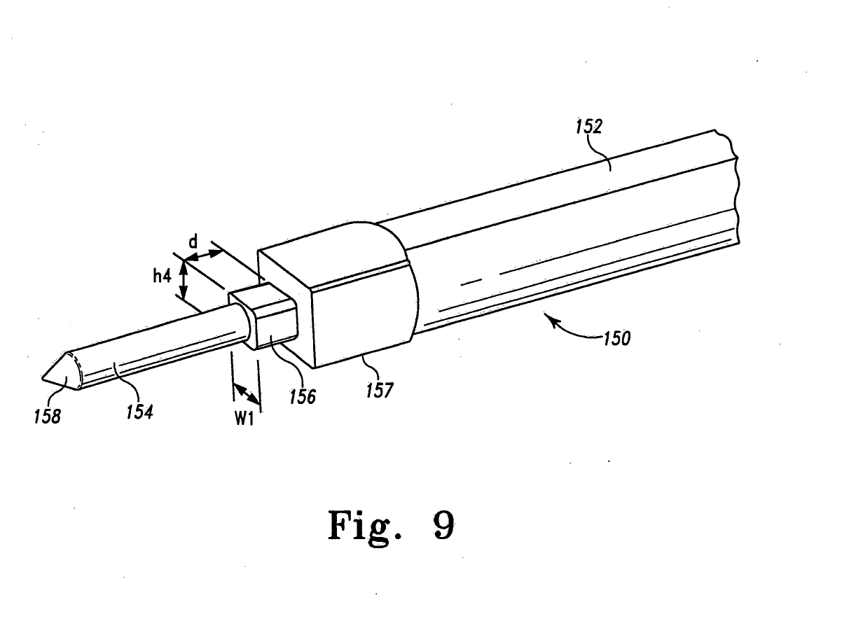

An insertional tool 150, a distal end coupling assembly of which is shown in FIG. 9, is engageable to device 100′ in through-opening 120 and hole 122 to facilitate insertion of device 100′ in the disc space. Insertional tool 150 includes an outer shaft 152 which houses a rotatable inner shaft 154. Outer shaft 152 includes a coupling member 156 at a distal end thereof which is received in through-opening 120. Coupling member 156 includes a height h4 and a width w1 that substantially corresponds to the height and width of through-opening 120 so that coupling member 156 is positioned in form fitting engagement therewith. Furthermore, coupling member 156 includes a longitudinal length d that corresponds to the depth of through-opening 120. This aligns the distal end of coupling member 156 with the proximal end of hollow interior 108, minimizing the space in hollow interior 108 occupied by the coupling assembly and increasing the space available for packing of bone graft or other material therein. A buttressing member 157 can be provided about shaft 152 proximally of coupling member 156 to abut trailing end 106 of cage body 102′ when assembled thereto.

Engaging end 158 extends distally from the distal end wall of coupling member 156, and includes a length extending distally from coupling member 156 that is sized to extend from the proximal end of hollow interior 108 to hole 122 at leading end 104. At least the distal portion of engaging end 158 can be threaded, and inner shaft 154 can be rotated to threadingly engage engaging end 158 in hole 122. Through-opening 120 can be non-circular, and coupling member 156 can be received in form fitting engagement therewith to prevent the outer shaft 152 from rotating or twisting in device 100 as engaging end 158 is engaged to device 100. This also facilitates unthreading engaging end 158 relative to hole 122. Other embodiments contemplate other engagement relationships between hole 122 and engaging end 158 other than a threaded engagement, such as interference fits, and outwardly biased or ball-detent mechanisms, for example.

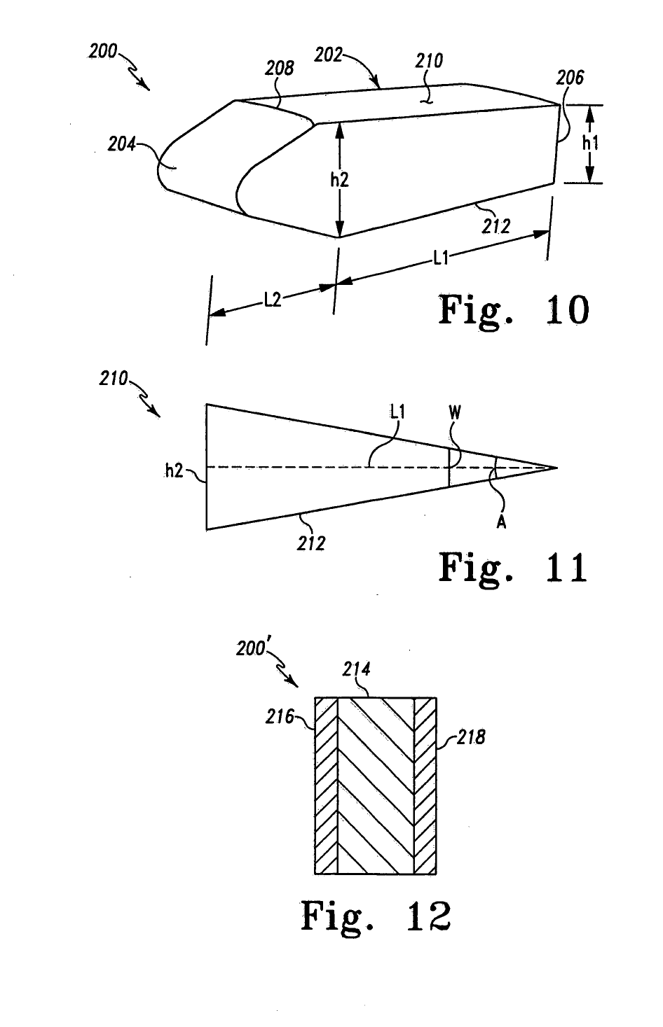

FIG. 10 shows another embodiment interbody device that is a bone implant made from dense cancellous bone, such as from the patella or vertebral body. Implant 200 includes a body 202 extending from a tapered leading end 204 to a trailing end 206. Body 202 includes upper and lower surfaces 210, 212 that slope away from one another from trailing end 206 to an apex 208. Body 202 includes a height h2 at apex 208 that is greater than height h1 at trailing end 206. Body 202 further includes a length L1 between trailing end 206 and apex 208. Body 202 also includes a tapered insertion tip having a length L2 and that tapers distally from apex 208 to leading end 204 to facilitate distraction of the disc space during insertion of body 202 between adjacent vertebrae.

FIG. 11 shows a diagram of the relationship between surfaces 210, 212 in which the surfaces form a slope angle A extending between height h1 at the trailing end and height h2 at the apex. In one specific embodiment, angle A is about 4 degrees, and length L1 ranges from 22 to 25 millimeters. Other lengths are contemplated based on the dimensions of the vertebral endplates to be supported by implant 200 and the desired fit therewith. The length L2 of the nose portion extending from apex 208 can be about 5 millimeters. The overall implant length can thus range from 27 to 30 millimeters in one specific embodiment. The heights of the implant can be selected to provide the desired disc space height.

In FIG. 12, there is shown a cross-section of a composite graft implant 200′ that can be similar to implant 200 discussed above. Implant 200′ includes a central region 214 of dense cancellous bone is sandwiched between outer layers of cortical bone 216, 218 to provide the implant with rigidity. The FIG. 12 embodiment increases the strength of the implant with the cortical bone to preserve the implant integrity as it is inserted into the disc space.

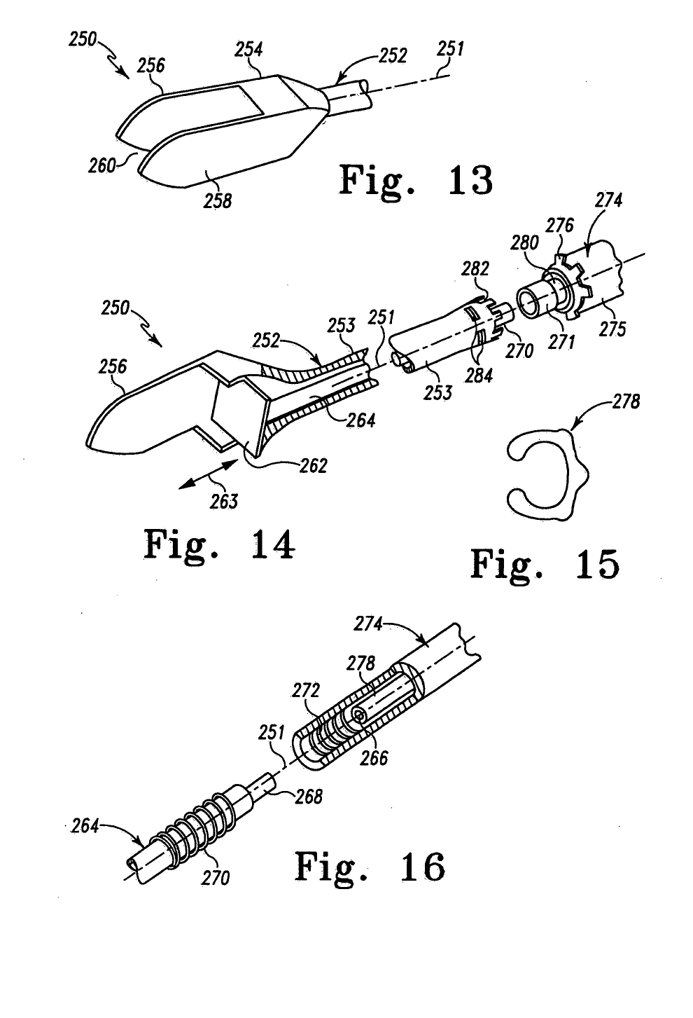

FIGS. 13-16 provide various views of an inserter instrument 250 having a distal engaging portion 254 with side flanges 256, 258 that extend along the sides of the implant. Engaging portion 254 is provided at a distal end of a shaft assembly 252 extending along longitudinal axis 251. The inserter instrument can be employed with, for example, the implants of FIGS. 5, 6, 10 or 12, and the side flanges 256, 258 act on the vertebral endplates to distract the disc space as the implant is inserted and to protect the implant from the insertion forces. The flanges 256, 258 form a space 260 therebetween to receive the implant, and flanges 256, 258 can be shaped to generally correspond to the shape of the implant along its sidewalls. In one form, the sidewalls of the implant and thus the flanges 256, 258 are parallel to one another between their leading and trailing ends and the flanges project from the upper and lower surfaces of the implant so that the flanges contact the adjacent vertebral bodies rather than the implant positioned therebetween during implant insertion.

Insertional tool 250 includes an implant pushing member 262 between flanges 256, 258 that is operable to move proximally and distally between flanges 256, 258 as indicated by arrows 263 and displace the implant forwardly from between flanges 256, 258 along axis 251. Pushing member 262 includes a distal plate like-member in contact with the trailing end of the implant positioned between flanges 256, 258 and a shaft 264 extending proximally therefrom through outer sleeve 253 of shaft assembly 252.

Outer sleeve 253 is coupled to a proximal handle portion 274 in end-to-end fashion. Handle portion 274 includes a coupling portion 271 extending distally from an enlarged gripping portion 275. Coupling portion 271 defines a groove 280 thereabout that receives a locking member 278, shown in FIG. 15. Locking member 278 includes an inwardly deflectable configuration and extends about and is axially retained in groove 280. Sleeve 253 can be positioned about coupling portion 271 so that projecting portions of locking member 278 are received in windows 284 of outer sleeve 253, axially restraining sleeve 253 on handle portion 274.

The proximal end of sleeve 253 can include a notched end 282 having a series of notches and axial extensions formed therein. Notched end 282 is configured so that respective ones of the radial extensions 276 of handle portion 274 are received in the notches about notched end 282. As discussed further below, rotation of handle portion 274 relative to outer sleeve 253 axially displaces pushing member 262. The locking member 278 can be resilient so that limited axial movement in groove 280 is permitted to allow limited axial displacement of sleeve 253 as handle portion 274 is rotated, allowing rotation of the radial extensions 276 past the respective adjacent axial extensions of notched end 282. Locking member 278 biases notched end 282 proximally back into interdigitating engagement with radial extensions 276. Accordingly, an audible and visual indication of advancement of pushing member 262 can be provided as handle portion 274 is rotated by the user.

As shown in FIG. 16, shaft 264 includes a proximal engaging portion 270 having external threads that can threadingly engage internal threads 272 in handle portion 274. Rotation of handle portion 274 is translated into axial movement of shaft 264, thus distally displacing pusher member 262 relative to flanges 256, 258. The threads can be provided with a pitch so that each click or set of clicks created upon rotation of handle portion 274 corresponds to a displacement distance of pushing member 262 between flanges 256, 258. Accordingly the user is provided an indication of the relative positioning of the implant between flanges 256, 258 even if the implant is not viewable since it is in the disc space. This can facilitate withdrawal of the insertion instrument from the disc space while the implant positioning in the disc space is maintained with pushing member 262. An extension 268 extending proximally from engaging portion 270 can be received in passage 266 of tube portion 278 to maintain alignment between handle portion 274 and shaft 264. Tube portion 278 can further act as a stop to limit the translational displacement between handle portion 274 and shaft 264.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

What is claimed is:

1. An interbody spinal implant system, comprising:
an interbody cage having a body extending along a longitudinal axis between a tapered leading end and an opposite trailing end, said body including opposite convexly curved upper and lower vertebral endplate contacting surfaces extending between said leading end and said trailing end and a rounded nose convexly curved between said upper and lower vertebral endplate contacting surfaces, said body further defining a hollow interior opening through each of said upper and lower vertebral endplate contacting surfaces, said body defining a hole extending along said longitudinal axis in said leading end in communication with said hollow interior and a through-opening in said trailing end in communication with said hollow interior; and
an insertional tool including an outer shaft and an inner shaft, said outer shaft including a coupling member at a distal end thereof non-rotatably engaged in said through-opening in said trailing end, said inner shaft being rotatably received in and projecting from said outer shaft, said inner shaft including a distal engaging end in engagement with said hole in said leading end of said body.

2. The system of claim 1, wherein said body of said interbody cage is made from titanium material.

3. The system of claim 1, wherein said body of said interbody cage is made from PEEK material.

4. The system of claim 1, wherein said distal engaging end of said insertional tool is threaded and threadingly engages said hole in said leading end of said body.

5. The system of claim 1, wherein said coupling member is received in said through-opening, and said inner shaft includes a length projecting from said coupling member, said length extending from said trailing end to said leading end of said body.

6. The system of claim 1, wherein said upper and lower vertebral endplate contacting surfaces each include a number of transverse grooves therein extending transversely to said longitudinal axis.

7. The system of claim 1, wherein said through-opening is non-circular and said coupling member is received in form fitting engagement with said through-opening.

9. The system of claim 1, wherein said body includes opposite sidewalls extending parallel to one another between said leading end and said trailing end and between said upper and lower vertebral endplate contacting surfaces, said through-opening having a width between said sidewalls that is greater than one half a width of said body between said sidewalls.

10. The system of claim 1, wherein said through-opening has a height that is greater than one half of a height of the body between said upper and lower surfaces at said trailing end.

11. The system of claim 10, wherein said through-opening has a width that is greater than one half of a width of the body between said opposite sidewalls thereof at said trailing end.

12. A method for inserting an interbody implant in a spinal disc space, comprising:
providing an interbody cage having a body extending along a longitudinal axis between a tapered leading end and a trailing end, said body including opposite convexly curved upper and lower vertebral endplate contacting surfaces extending between said leading end and said trailing end, said body further defining a hollow interior opening through each of said upper and lower vertebral endplate contacting surfaces, said body defining a hole in said leading end in communication with said hollow interior and a through-opening in said trailing end in communication with said hollow interior;
providing an inserter including an outer shaft and an inner shaft, said outer shaft including a coupling member at a distal end thereof;
non-rotatably engaging said coupling member in said through-opening in said trailing end;
engaging said inner shaft with said hole in the leading end of the body of the interbody device;
re-capitulating the spinal disc space with the leading end of the body of the interbody cage; and
inserting the interbody cage into the spinal disc space with said convexly curved upper and lower vertebral endplate contacting surfaces in contact with respective adjacent ones of upper and lower vertebral endplates.

13. The method of claim 12, wherein said coupling member defines a shape received in form fitting engagement in said through-opening at said trailing end wall, said coupling member including a distal end wall that aligns with a proximal end of said hollow interior when positioned in said through-opening, and wherein said inner shaft extends from said distal end of said coupling member to engage said hole at said leading end of said body.

14. The method of claim 13, further comprising packing the hollow interior with bone growth material with the inner shaft extending through the hollow interior.

15. An interbody spinal cage, comprising:
a body including opposite sidewalls extending along a longitudinal axis between a tapered leading end and an opposite trailing end, said body including opposite convexly curved upper and lower vertebral endplate contacting surfaces extending between said leading end and said trailing end and a rounded nose at said leading end extending between said vertebral endplate contacting surfaces, said body further defining a hollow interior opening through each of said upper and lower vertebral endplate contacting surfaces, said body defining a hole in said leading end in communication with said hollow interior and a through-opening in said trailing end in communication with said hollow interior, wherein said through-opening has a height that is greater than one half a height of said body between said upper and lower vertebral endplate contacting surfaces at said trailing end and said through-opening has a width that is greater than one half a width of said body between said opposite sidewalls thereof at said trailing end.

16. The cage of claim 15, wherein said body is made from titanium material.

17. The cage of claim 15, wherein said body is made from PEEK material.

18. The cage of claim 15, wherein said upper and lower vertebral endplate contacting surfaces include a number of transverse grooves therein extending transversely to said longitudinal axis.

19. The cage of claim 15, wherein said opposite sidewalls of said body are parallel.

20. The cage of claim 15, further comprising a coupling assembly engageable in said hollow interior of said body, said coupling assembly including a coupling member positionable in said through-opening in form fitting engagement with said through opening, said coupling member including a distal end wall aligned with a proximal end of said hollow interior, and said coupling assembly further including an engaging member within said coupling member and extending distally from said distal end wall of said coupling member, said engaging member extending through said hollow interior and engaging said hole at said leading end of said body.

21. An interbody spinal implant system, comprising:
an interbody implant having a body extending along a longitudinal axis, said body further including upper and lower surfaces extending between a leading end and an opposite trailing end of said body, said upper and lower surfaces defining a maximum height at a location between said leading end and said trailing end, wherein said upper and lower surfaces extend toward one another from said location toward said leading end and toward said trailing end, said body further including opposite parallel sidewalls from said leading end to said trailing end; and
an insertional tool including a shaft assembly with an outer shaft and an inner shaft and a distal engaging portion extending from said shaft assembly, wherein said inner shaft is movable proximally and distally relative to said outer shaft to engage a distal end of said inner shaft with said body of said implant and said distal engaging portion extends along said body of said implant from said trailing end to said leading end of said body.

22. The system of claim 21, wherein said leading end of said body is convexly rounded between said upper and lower surfaces.

23. The system of claim 21, wherein said body of said implant is made of titanium material.

24. The system of claim 21, wherein said body of said implant is made from PEEK material.

25. The system of claim 21, wherein said distal end of said inner shaft includes a pushing member that is movable distally to displace said implant along said distal engaging portion.

26. The system of claim 25, wherein said distal engaging portion includes a pair of opposite flanges extending along said sidewalls of said body of said implant between said leading end and said trailing end and said pushing member is located between said opposite flanges.

27. The system of claim 26, wherein said opposite flanges project from said upper and lower surfaces of said body of said implant.

28. The system of claim 27, wherein said body of said implant is made from bone material.

29. The system of claim 26, wherein said pushing member is a plate that extends transversely to said inner shaft.

30. The system of claim 21, wherein said location of said maximum height is positioned closer to said leading end than said trailing end.
